(19) United States
(12) Patent Application Publication
(10) Pub. No.: US 2004/0193027 A1
(43) Pub. Date: Sep. 30, 2004

(54) Apparatus and method for the non-invasive measurement of parameters relating to biological tissues by spectroscopy, in particular with infra-red light

(76) Inventors: Mario Ettore Giardini, Cava Manara (IT); Giovanni Guizzetti, Pavia (IT)

Correspondence Address: SUGHRUE MION, PLLC, 2100 PENNSYLVANIA AVENUE, N.W., SUITE 800, WASHINGTON, DC, 20037, US

(21) Appl. No.: 10/486,132

(22) PCT Filed: Aug. 5, 2002
(86) PCT No.: PCT/IB02/03080

(30) Foreign Application Priority Data
Aug. 9, 2001 (IT) PD01A000205

(51) Int. Cl. A61B 5/00

(52) U.S. Cl. 600/323

(57) ABSTRACT

Apparatus for the non-invasive measurement of parameters relating to a biological tissue by spectroscopy, in particular with infra-red light, is described and comprises a plurality of light sources each of which can emit a light signal with one or more predetermined wavelengths, towards the tissue, a light detector for detecting a light signal transmitted by the tissue as a result of the illumination by the sources, and driving means for the light emitted by the sources, for modulating the light signal emitted by each of the sources, the apparatus further comprising processing means to which the driving means are coupled in order to control the modulation of the light signals emitted by the sources in a “spread-spectrum” manner. A method for the non-invasive measurement of parameters relating to a biological tissue by spectroscopy, in particular with infra-red light, is also described.

The present invention relates to apparatus for the non-invasive measurement of parameters relating to biological tissues by spectroscopy, in particular with infra-red light, according to the preamble to main claim 1. A further subject of the invention is a method for the measurement of these parameters by spectroscopy.

Infra-red light spectroscopy has many applications in the technical field of non-invasive diagnostics, for example, in the determination of the oxygenation or perfusion of tissues and in the detection of breast tumours. Infra-red spectroscopy is also used in sports, at competitive level, where a knowledge of predetermined parameters such as the oxygenation of the muscles or the percentage of fat in the tissues is necessary to determine the correct training program for the sportsperson.

For example, this technique enables the degree of oxygenation of the tissues to be detected since haemoglobin, which is responsible for this oxygenation, has a different infra-red absorption spectrum according to whether it is in an oxygenated or deoxygenated state. Moreover, in this region of the spectrum, haemoglobin is the dominant optical absorber so that, if the biological tissue in question is illuminated with infra-red light at predetermined wavelengths at which the absorption coefficients of oxygenated and of deoxygenated haemoglobin are known, and the light scattered, transmitted or back-scattered by the tissue is detected, the ratio between the two quantities of haemoglobin present in the tissue of interest, that is, a measurement of the oxygenation of the tissue, is obtained.

In known infra-red spectroscopy apparatus, a plurality of infra-red light sources and one or more detectors of the light transmitted or scattered by the organ or tissue to be analyzed are generally used. The signal from the detectors therefore has to be processed appropriately to obtain the parameter of interest. For this purpose, it is necessary for the components due to the various sources to be identifiable in the signal detected. However, the signal reaching the detector is very often extremely weak and affected by noise consisting of interference by electromagnetic waves which are present in the same environment.

To prevent this problem, known apparatus is therefore provided with large screens which render it heavy and bulky as well as expensive. Moreover, the processing of the signals affected by the noise requires filtering methods which, irrespective of whether they are implemented by analog or digital means, are generally complex.

The technical problem underlying the present invention is that of providing apparatus and a method for the non-invasive measurement of parameters of biological tissues by spectroscopy which are designed structurally and functionally to overcome the problems discussed with reference to the prior art mentioned.

This problem is solved by the present invention by means of apparatus and a method according to the appended claims.

The characteristics and the advantages of the invention will become clearer from the description of a preferred embodiment thereof, described by way of non-limiting example, with reference to the appended drawings, in which:

FIG. 1 is a schematic plan view of the measurement apparatus according to the invention,

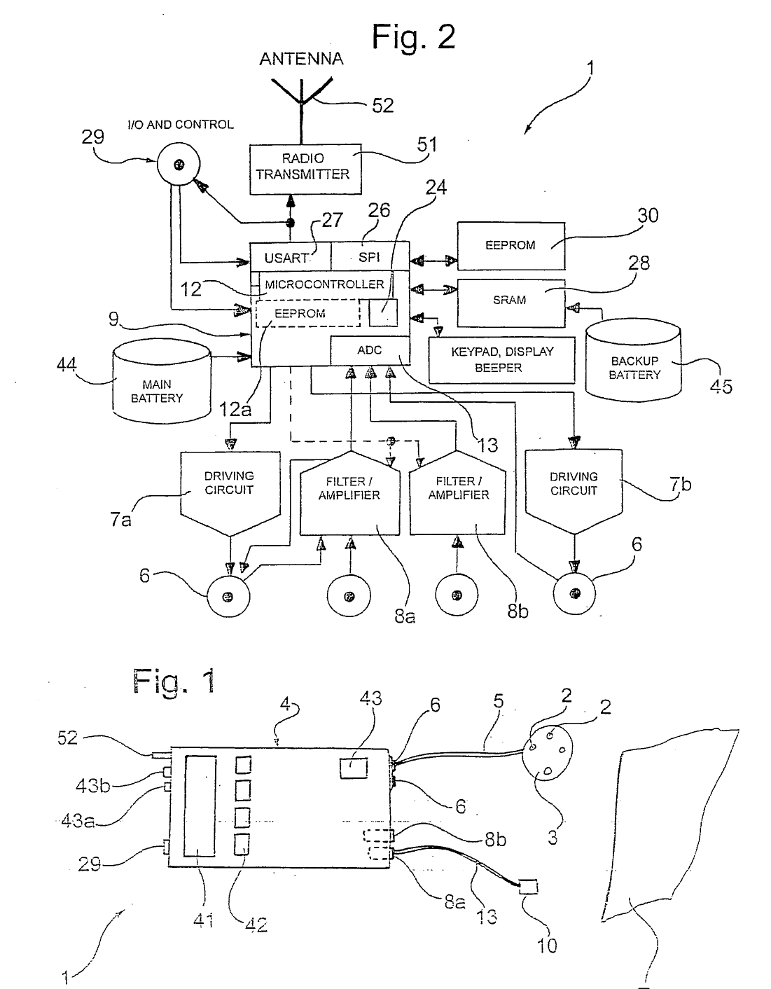

FIG. 2 is a block diagram of the measurement apparatus of FIG. 1,

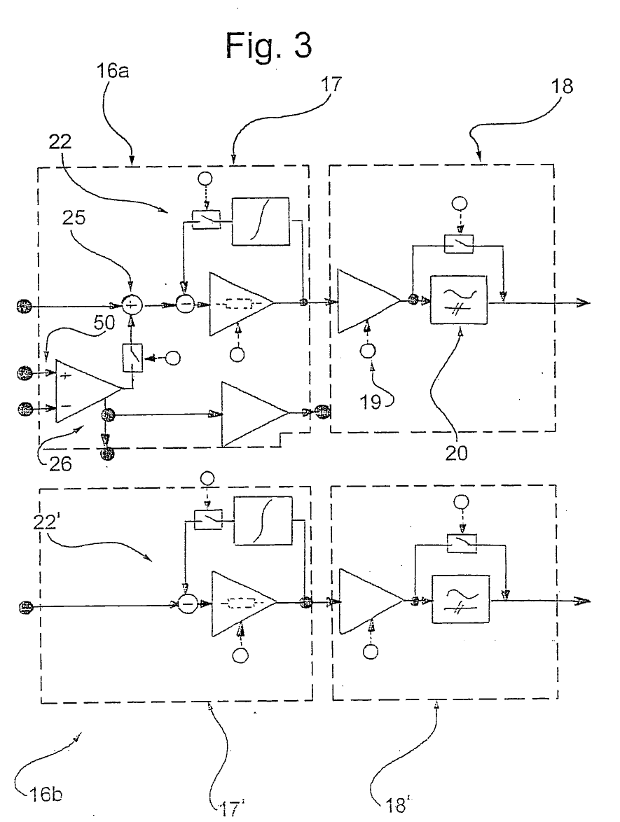

FIG. 3 is a block diagram of a detail of the apparatus of FIG. 2, and

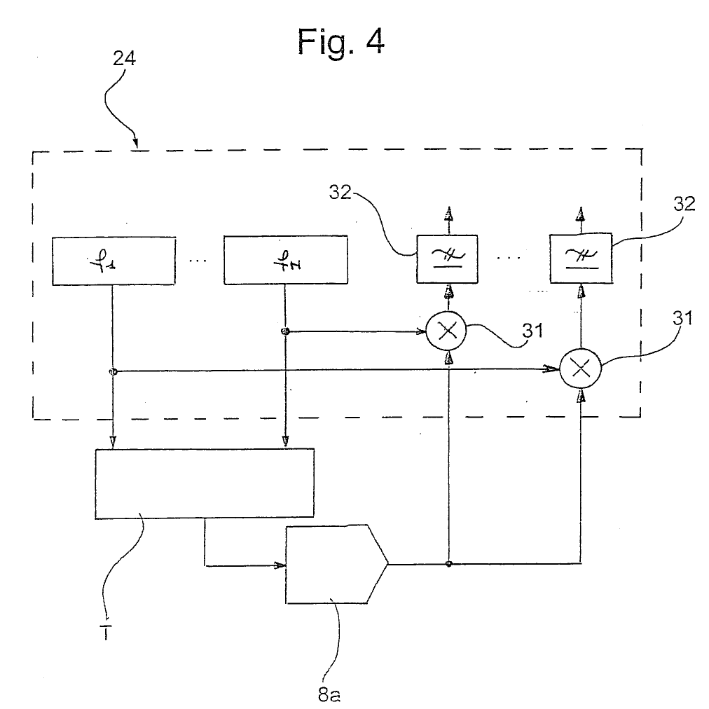

FIG. 4 is a diagram showing one of the steps of the measurement method according to the invention.

With reference initially to FIG. 1, apparatus for the non-invasive measurement of parameters of biological tissues by infra-red light spectroscopy, in particular an oximeter, is generally indicated 1.

The apparatus 1 comprises a plurality of infra-red light sources 2, in particular, two groups of four LEDs with emission peaks at 660 nm, 700 nm, 850 nm, and 880 nm. Each group is mounted on a substrate 3 of plastics material and connected, by means of a screened cable 5, to a current driver with four channels 6, housed in a box-like housing 4. The sources 2 can illuminate a biological tissue T, the degree of oxygenation of which is to be obtained, with infra-red light in a programmed sequence, as described in detail below. The number of sources 2 usable by the apparatus 1 during a measurement may be variable and determined by the type of measurement to be made.

The apparatus 1 also comprises driving means 7a, 7b, which are subject to processing means 9, and which can bring about a modulation of the signal emitted by each of the sources 2, describable by a function f, in which n=1, . . . , N, where N is equal to the number of sources 2 used in the measurement, in accordance with parameters configured in the processing means 9.

The apparatus 1 further comprises, in the box-like housing 4, two independent receiving channels 8a, 8b, to each of which a light detector 10, in particular a photodiode PIN, for receiving the light transmitted through the tissue T, can be connected by means of a screened cable 13.

Two further non-amplified analog channels 43a, 43b are also accessible from the box-like housing 4 for the connection of further auxiliary sensors (not shown).

The receiving channels 8a, 8b, which can filter and amplify the signal coming from the detector 10 as described in detail below, are connected electrically to an analog/digital converter 13 (ADC) configured so as to be activated by the processing means 9. The digital signal output thereby is processed by a microcontroller 12 included in the processing means 9 and in turn including an internal memory 12a.

Both of the receiving channels 8a, 8b comprise programmable amplification and filter means, generally indicated 16a and 16b, respectively, a block diagram of which is shown in FIG. 3.

The amplification and filter means 16a comprise a structure with two stages 17, 18, the first stage 17 being configurable alternatively as a high-sensitivity transconductance amplifier for direct connection to a photodiode, or as a voltage amplifier for connection to a preamplified detector. The second stage 18, which is in series with the first 17, comprises a variable-gain voltage amplifier 19, controllable by software, followed by a high-pass filter 20 which can be excluded, also connected to the ADC 13.

The first stage 17 includes adding means 25 for adding to the input signal coming from the detector 10 signals coming from a high-impedance differential amplifier 26 comprising input connections 50.

A high-pass filter 22 in integration-subtraction mode is coupled to the adding means 25, in a manner such that it can be excluded, to compensate for direct-current background signals which may be a few orders of magnitude greater than the signal detected by the detectors 10.

The amplification and filter means 16b comprise a structure very similar to that of the means 16a, also comprising a two stage-structure with a first stage 17 comprising a high-pass filter 22 in integration-subtraction mode, similar to the high-pass filter 22, and a second stage 18 formed in a similar manner to the second stage 18 of the means 16a.

The processing means also comprise demultiplexing means 24 for distinguishing the contribution of each individual source 2 to the digital signal output by the ADC 13. The demultiplexing means 24 in turn comprise means 31 for multiplying the digital signal and means 32 for integrating the digital signal.

Also connected to the processing means 9 are a mass memory unit 28 for the storage of the data processed thereby, and a plurality of peripheral units comprising a universal synchronous-asynchronous serial transmitter/receiver (USART) 27 and an SPI interface 26.

A serial I/O control port 29 is also available in the box-like housing 4 for the programming of the processing means so that further modifications or improvements of the functionality of the apparatus 1 can be made without disassembly of the apparatus. The serial port 29 is also connected to the peripheral USART and to a radio transmission station 51 comprising an antenna 52.

The internal EEPROM memory 12a can store a program for configuring the various stages of the measurement operation and the respective protocol. Further memory units 30, preferably of the SRAM type, are connected to the processing means to increase its data-holding capacity.

A LED display 41, a keypad 42, and a beeper 43 are also incorporated in the box-like housing.

The apparatus 1 is supplied by a battery 44 and also comprises a backup battery 45.

In order to perform a measurement of the degree of oxygenation of the tissue T, the measurement method according to the invention provides for the simultaneous emission, by each of the sources 2 (for example, four sources 2), of an infra-red light signal with a predetermined wavelength. The driving means 7a impart to the signal emitted by each source 2 a modulation, set in the processing means 9, so that a modulation function f, the characteristics of which will be described in detail below, is coupled with each nth source (where n is from 1 to N and N is equal to the number of sources used).

The processing means 9 are programmed and configured by means of a computer, not shown, connected to the apparatus 1 by means of the serial port 29. In particular, within the memory 12a, there is a table in which the values of the predetermined modulation functions fₙ are given. The measurement protocol program, which is also resident in the memory 12a, may also be modified.

The method then provides for the intensity of light transmitted by the tissue T to be detected by the light detector 10 and for the signal S thus obtained, which is equal to

$$S = {\sum }_{n = 1 , … , N} {a}_{n} {f}_{n}$$

where αₙ are the responses associated with each individual nth source 2, to be sent to the preselected receiving channel, in this case 8b.

In the receiving channel 8b, the signal S is filtered and amplified appropriately as described above and is then sent to the ADC converter 13, by which the signal is converted from analog to digital. The signal is thus sent from the ADC to the microcontroller 12 in the form of a digital signal S′.

A step is then provided for demultiplexing the signal S′ thus processed, that is, an association is formed between each component constituting the signal S′ and the respective source which produced that component. In particular, to perform the demultiplexing, the multiplication means 31 multiplies the signal S′ by each of the functions fₙ independently, and each nth signal thus obtained is then advantageously integrated over a suitable time interval by the integration means 32. By virtue of the particular structure of the preselected modulation functions, a number N of components Sₙ equal to the number of sources is thus obtained, each component S, being associated with the respective nth source.

By virtue of the transformation of the signal detected into a digital signal, the demultiplexing step can be represented by simple algebraic operations which can be performed digitally by means of a program entered in the microcontroller 12, and the multiplication means 31 and the integration means 32 therefore represent nothing other than different steps of a program. In order to perform these operations, the table containing the values adopted by the functions fₙ is periodically interrogated by the microcontroller 12, simultaneously for all of the sources 2, in order to determine their state. Alternatively, an analog circuit, in which the integration means 32 comprise a low-pass filter (not shown), may be provided.

The N components Sₙ thus obtained are processed by the microcontroller 12 by means of known algorithms to obtain a measurement of the degree of oxygenation of the blood. The data obtained by the measurement process are then stored in the memory 12a of the microcontroller 12 and optionally downloaded into an external mass memory by means of the serial I/O connection. Alternatively, the data collected may be transmitted by radio, by means of the transmission station 51 and the antenna 52.

Because of the combined absorption and scattering effect, the light detected by the detector 10 is greatly attenuated in comparison with the light emitted by the sources 2. The signal detected is therefore very sensitive to noise, especially electromagnetic noise. The introduction of the modulation into the signal emitted by the sources 2 reduces the effect of the electromagnetic noise in the signals detected. In particular, the type of modulation used in the method of the present invention is effective for narrow-band noise and also enables the contribution of each individual source to the signal detected to be distinguished.

A modulation method used in the known apparatus, known as “lock-in” modulation, is not effective in improving the signal-to-noise ratio since the most powerful sources of noise which are usually present in hospital environments emit in the same frequency band in which most of the electronic components operate, so that a lock-in modulation (in which the functions fₙ are essentially sinusoidal) would not lead to any improvement.

According to a particular feature of the invention, the functions fₙ are selected in a manner such as to have behaviour similar to that of a noise signal, that is, these functions have a low cross-correlation both with one another and with random or periodic noise sources. This type of modulation is called “spread-spectrum”, that is broadened-spectrum, modulation, as will become clear from the following example.

In the demultiplexing step, the nth component of the signal S due to the nth source is calculated by the multiplication means 31 and the integration means 32,

$${S}_{n} = \int {f}_{n} S ⅆ t = {\sum }_{m = 1 , … , N} \int {a}_{m} {f}_{n} {f}_{m} ⅆ t = {a}_{n} \int {f}_{n}^{2} ⅆ t$$

The latter equality is attributable to the fact that the cross-correlation of the modulation functions has been selected as zero, as shown above, and therefore only the nth element of the sum is other than 0. In the event of contamination by noise, the only type of noise which would alter this demultiplexing process, that is, which would affect the value of the integral calculated above, would be a noise very close to the function fₙ. However, if the same fₙ has a form similar to that of a noise signal and, by definition, independent noises show a high autocorrelation and a low cross-correlation, it is very improbable that this similarity will occur. In the demultiplexing process, by means of multiplication by fn, the spectrum of every other noise source is therefore “spread” over the entire frequency interval occupied by fₙ and the integration thus reduces the amplitude of the noise to minimal levels.

In particular, in the method according to the invention, functions oscillating between 0 and 1 in a pseudo-random sequence with low cross-correlation are preselected as modulation functions fₙ. Moreover, for all of the N sources, these sequences are periodic with a common period and the transitions in value between 0 and 1 are performed at moments which are multiples of a common time interval. Functions which satisfy these specifications are, for example, the “gold codes” developed by Magnavox Corporation.

The apparatus and the method described relate to the measurement of the oxygenation of a tissue or of an organ, but the same method and apparatus can also be applied to the measurement of further parameters simply with the use of a different algorithm for processing the components Sₙ obtained as signals output by the demultiplexing means 24. This apparatus may therefore be, for example, mammography apparatus, or apparatus for measuring the percentage of fat in the tissues.

The invention thus solves the problem posed, affording many advantages over known solutions.

A first advantage is that, since the operations to control the modulation of the sources and the subsequent demultiplexing of the signal are simple operations which can be performed by a program, the hardware of the measurement apparatus of the invention is very limited, simplifying the production of the apparatus.

In addition, by virtue of the “spread-spectrum” signal-modulation method used, contamination of the detected signal by noise is extremely difficult and bulky screening is not therefore necessary.

The two advantages listed above mean that the measurement apparatus produced is compact and light, in other words portable, and is thus suitable for use in non-specific environments such as, for example, on athletics training grounds.

1. (Oringnal) Apparatus for the non-invasive measurement of parameters relating to a biological tissue by spectroscopy, in particular with infra-red light, comprising:
a plurality of light sources, each of which can emit a light signal with one or more predetermined wavelengths, towards the tissue,
a light detector for detecting a light signal transmitted by the tissue as a result of the illumination by the sources, and
driving means for the light emitted by the sources, for modulating the light signal emitted by each of the sources, characterized in that it further comprises:
processing means to which the driving means are coupled in order to control the modulation of the light signals emitted by the sources in a “spread-spectrum” manner.

2. (Oringnal) Apparatus according to claim 1 in which the processing means comprise demultiplexing means, connected to the light detector, in order to determine the contribution of each of the sources to the signal detected by the detector and to output a plurality of signals proportional to the contributions.

3. (Oringnal) Apparatus according to claim 2 in which the demultiplexing means comprise multiplication means for independently multiplying the signal detected by each of the light signals emitted and outputting a number of multiplied signals equal to the number of sources.

4. (Oringnal) Apparatus according to claim 3 in which the demultiplexing means comprise integration means for integrating the multiplied signals over a suitable time interval and obtaining the contributions of each of the sources to the signal detected by the detector.

5. (Currently Amended) Apparatus according to one or more of the preceding claims 1 in which the light signals emitted are functions oscillating between 0 and 1 in a pseudo-random sequence with low cross-correlation.

6. (Currently Amended) Apparatus according to one or more of the preceding claims 1 in which the light signals emitted are gold codes.

7. (Currently Amended) Apparatus according to one or more of the preceding claims 1, comprising means, interposed between the detector and the processing means, for amplifying and filtering the signal detected.

8. (Currently Amended) Apparatus according to any one of claims 2, to 7, comprising a memory for storing numerical values of the contributions of each of the sources to the signal detected by the detector.

9. (Currently Amended) Apparatus according to any one of claims 2, to 8 in which the demultiplexing means comprise a program which can be executed by the processing means.

10. (Currently Amended) Apparatus according to one or more of the preceding claims 1 in which the parameter is the oxygenation of the biological tissue.

11. (Currently Amended) Apparatus according to one or more of the preceding claims 1 in which the parameter is the perfusion and/or the vascularization of the biological tissue.

12. (Oringnal) A method for the non-invasive measurement of parameters relating to a biological tissue by spectroscopy, in particular with infra-red light, comprising the steps of:
emitting a plurality of light signals towards the tissue by means of a plurality of sources,
detecting a light signal transmitted by the tissue, and
driving each of the plurality of light signals emitted by the sources, by means of driving means,
characterized in that it further comprises the step of:
rendering the driving means subject to processing means,
controlling the driving means by means of the processing means in order to modulate the light signals emitted, in a “spread-spectrum” manner.

13. (Oringnal) A method according to claim 12, further comprising the step of demultiplexing the signal detected to determine the contribution of each of the sources to the signal detected.

14. (Oringnal) A method according to claim 13 in which the demultiplexing step comprises a step of independent multiplication of the detected signal by each of the light signals emitted, and the output of a number of multiplied signals equal to the number of sources.

15. (Oringnal) A method according to claim 14 in which the demultiplexing step comprises a step of integrating the multiplied signals over a suitable time interval and obtaining the contributions of each of the sources to the signal detected by the detector.

16. (Currently Amended) A method according to any one of claims 12 to 15, comprising the step of amplifying and filtering the signal detected by the detectors.

17. (Currently Amended) A method according to one or more of claims 13 to 16, comprising the step of storing numerical values of the contributions of each of the sources to the signal detected by the detector, in a memory.
